(19) United States
(12) Patent Application Publication
(10) Pub. No.: US 2024/0122729 A1
(43) Pub. Date: Apr. 18, 2024

(54) PROSTHETIC DIGIT

(71) Applicant: General Articulating Point, LLC, Rochester, MN (US)

(72) Inventor: Brandon Sampson, Pine Island, MN (US)

(21) Appl. No.: 18/489,524

(22) Filed: Oct. 18, 2023

Related U.S. Application Data
(60) Provisional application No. 63/417,258, filed on Oct. 18, 2022.

(51) Int. Cl. A61F 2/58; A61F 2/68

(52) U.S. Cl. CPC A61F 2/586; A61F 2/68; A61F 2002/5043

(57) ABSTRACT

A prosthetic digit can include a proximal segment connected to an intermediate segment about a first axis of rotation and a distal segment connected to the intermediate segment about a second axis of rotation. A linkage can be connected to the proximal segment about a third axis of rotation and can couple the proximal segment to the distal segment. The linkage can be configured such that a distal end of the distal segment to rotate toward the proximal segment about the second axis of rotation when the intermediate segment is rotated in a palmar direction about the first axis of rotation. A unidirectional lock can allow the intermediate segment to rotate about the first axis of rotation in a palmar direction but not in a dorsal direction. A release mechanism can release the unidirectional lock to allow the intermediate segment to rotate in the dorsal direction.

### CROSS-REFERENCE

This application claims the benefit of U.S. Provisional Patent Application No. 63/417,258, filed Oct. 18, 2022, the entire contents of which are incorporated herein by reference.

### TECHNICAL FIELD

Aspects of the disclosure relates to prostheses to replace one or more digits of a human hand.

### BACKGROUND

Partial hand loss is a common amputation often impacting a person's ability to perform many tasks, such as recreational or professional tasks or life functions, such as dressing, eating, or preparing food. Prosthetic intervention can be used to restore a forceful grasp capability of the hand, such as between the thumb and the fingers, such that objects can be grasped even in the case of a partial hand loss. However, because digits of the hand have such wide-ranging functions, some prosthetic interventions may not effectively restore the functionality lost.

Three broad categories of available prostheses include cosmetic, passive, and active prostheses. Cosmetic prostheses generally aim to resemble original anatomy, but often include minimal functionality apart from aesthetics. Passive prostheses include those that are not actively driven, but often include one or more movable joints, for example, mimicking joints of a digit. Such devices can be adjustable to several configurations to mimic postures of the replaced anatomy. Active prostheses are generally driven by the body or other power source (e.g., electrical power). However, active prostheses are often complex, expensive, and generally have low power output.

### SUMMARY

In general, the present disclosure is directed to prosthetic digits for the human hand and associated systems and techniques involving a prosthetic digit. In some example, a prosthetic digit according to the disclosure incudes multiple segments joined together a respective rotational axis to replication different bone and joint segments of the native digit being replaced by the prosthetic component. For example, the prosthetic digit may include a proximal segment, a distal segment, and an intermediate segment positioned between the proximal and distal segments. The three segments can be rotationally coupled to each other around different axes of rotation. In some configurations, the proximal segment of the prosthetic digit may be configured to partially or fully replace a metacarpal bone of the hand while the intermediate and distal segments of the prosthetic digit may be configured to partially or fully replace phalange bones of the hand (e.g., a proximal phalanx, intermediate or middle phalanx, and/or distal phalanx).

A prosthetic digit according to the disclosure may include movable joints, mimicking joints of a native digit, along with spring extension and locking retraction functionality. In use, a wearer can articulate the distal segment of the prosthetic digit relative to the intermediate segment of the prosthetic digit and/or articulate the intermediate segment of the prosthetic digit relative to the proximal segment of the prosthetic digit. In some implementations, the prosthetic digit includes a linkage coupling the proximal segment to the distal segment such that, when the intermediate segment is articulated relative to the proximal segment, the distal segment articulates a corresponding amount relative to the intermediate segment via the linkage between the proximal and distal segments. In either case, the prosthetic digit may include a locking system to lock a relative amount of articulation between the different segments of the prosthetic digit, e.g., thereby setting the relative angles between the different segments of the digit to a positioned desired by the user based on the task being undertaken. The lock can be releasable by the user, with one or more springs in the prosthetic digit causing the different segments of the digit to articulate to their extended position upon release of the lock.

A prosthetic digit according to the disclosure may provide good functionality for a wearer without the cost and complexity of a more complex prosthetic digit. In some applications, a wearer may be fitted with a prosthetic digit according to the disclosure and the prosthetic digit may provide all the functionality required by the wearer to suitably restore their life functions. In some applications, a prosthetic digit according to the disclosure may provide an intermediate level of functionality less than that provided by a more complex active prosthetic digit. In practice, an amputee seeking a more complex active prosthetic digit may have to wait a significant amount of time before receiving a more complex active prosthetic digit. For example, the amputee may have to satisfy rigorous insurance requirement to demonstrate eligibility for the complex active prosthetic digit and, even then, wait for fabrication and fitting of their new digit. In these situations, a prosthetic digit according to the disclosure may be used to help restore life function during a period while the amputee is waiting for a more complex active prosthetic digit and then replaced with that digit once available.

Independent of the specific circumstances under which an amputee acquires a prosthetic digit according to the disclosure, the prosthetic digit can provide a variety of different functional features. In some examples, the digit includes a locking system that includes a unidirectional lock and a release mechanism. The locking system can be configured to allow an intermediate segment of the digit to rotate in a first direction (e.g., palmar direction) about a first axis of rotation but lock the intermediate segment from rotating in an opposed direction (e.g., a dorsal direction). In some examples, the locking system includes a pawl and ratchet surface. For example, the unidirectional lock may provide a ratchet surface positioned on the intermediate segment and a pawl configured to engage the ratchet surface and prevent rotation of the intermediate segment in the dorsal direction. A lever can be spring biased in favor of the pawl engaging the ratchet surface and can be maniuplable to overcome the spring bias and disengage the pawl from the ratchet surface. In user, the wearer can depress a first end of the lever, disengaging the pawl from the ratchet surface and allowing a biasing force (e.g., spring) to cause the intermediate segment to move from a locked articulated position to a fully extended position.

In some configurations, the prosthetic digit also includes an anchor. The anchor can be operatively connected to the proximal segment of the prosthetic digit. For example, the proximal segment may be configured to be removably coupled to the anchor in a plurality of rotational positions. For instance, a system including the digit may include a wearable support that can be configured to be fastened to one or both of a wearer's hand or writ. The wearable support can include the anchor affixed thereto to facilitate attachment of the prosthetic digit to the wearable support via the anchor.

The details of one or more examples are set forth in the accompanying drawings and the description below. Other features, objects, and advantages will be apparent from the description and drawings, and from the claims.

### BRIEF DESCRIPTION OF THE DRAWINGS

FIG. 1 shows an illustration of the skeletal anatomy of a hand.

FIG. 2A shows an example prosthetic digit.

FIG. 2B shows an alternate view of the prosthetic digit shown in FIG. 2A.

FIGS. 2C and 2D show side views of a prosthetic digit in different rotational positions.

FIGS. 3A, 3B, and 3C show cross-sectional views of a prosthetic digit and an anchor according to some embodiments.

FIG. 4 shows an exploded view of a prosthetic digit according to some embodiments.

FIG. 5 shows a plurality of views of an anchor and a proximal segment of a prosthetic digit configured to attach to the anchor.

FIG. 6 shows a plurality of views of an example prosthetic digit in an extended configuration and attached to an anchor.

FIG. 7 shows a plurality of views of an example prosthetic digit in a retracted configuration and attached to an anchor.

FIG. 8 shows an example wearable support configured to be attached to a wearer.

FIG. 9 shows an example wearable support attached to a wearer's anatomy and supporting a plurality of prosthetic digits.

### DETAILED DESCRIPTION

The present disclosure is generally directed to a prosthetic finger (referred to as a prosthetic digit) configured to replace a finger or thumb in partial or full hand loss conditions. The prosthetic digit can be composed of multiple segments rotatably coupled together to define joints between the different segments. The prosthetic digit can include one more springs and/or locks to control and set the positioning of different segments of the prosthetic digit relative to each other. In use, the wearer may press a distal portion of the prosthetic digit one direction (e.g., a palmer direction) by pressing the distal portion of the digit against a stationary surface. This can cause the distal portion of the digit to rotate relative to a proximal portion of the digit, e.g., cause the distal portion to retract or curl. A locking system can hold the rotated position of the distal portion relative to the proximal portion. The wearer can release the lock, allowing one or more springs in the digit to push the retracted distal portion of the digit back to an extended (unretracted) position. The prosthetic digit can operate without electrical power and may be devoid of a battery, electrical wiring, and/or other electrical control features.

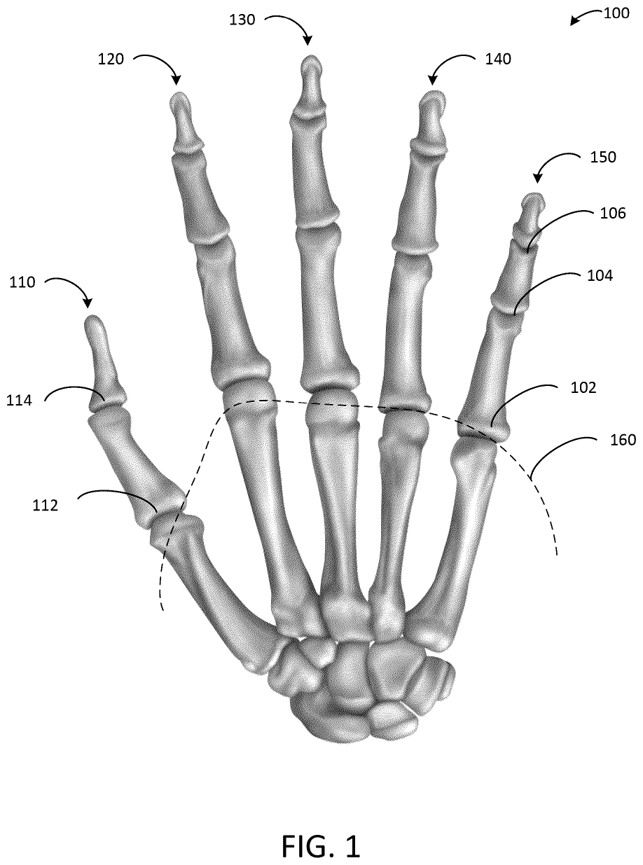

To further understand example prosthetic digits according to the disclosure, the anatomy of the hand will first be described with respect to FIG. 1. FIG. 1 shows an illustration of the skeletal anatomy of a hand 100. The hand 100 includes five digits 110, 120, 130, 140, 150, with digit 110 corresponding to a thumb. Broken line 160 shows an approximate location of the palm of the hand, with digits 110, 120, 130, 140, 150 extending therefrom. Thumb 110 includes a metacarpophalangeal (MCP) joint 112 and an interphalangeal (IP) joint 114. In the illustrated example, each of remaining digits 120, 130, 140, 150 include an MCP joint 102, a proximal interphalangeal (PIP) joint 104, and a distal interphalangeal (DIP) joint 106, as labeled in FIG. 1 on digit 150.

During movement of one or more digits, a person can move a digit in a palmar direction, in which one or more joints bend and the digit moves closer to the palm, or can extend a digit in a dorsal direction, moving the digit away from the palm. Palmar motion of each of digits 110, 120, 130, 140, 150 can bring the digits inward to close a fist, bending MCP joints 102, PIP joints 104, and DIP joints 106 of digits 120, 130, 140, 150 and MCP joint 112 and IP joint 114 of digit 110. Opening the hand from a first includes moving each such joint in a dorsal direction.

Some prostheses described herein can be used to replace a digit lost at or proximal of the MCP joint 102, 112. Some such prostheses can provide palmar movement of the prosthesis and provide resistance against movement in the dorsal direction so as to enable grasping pressure using the prosthesis and remaining anatomy, such as between a thumb and the prosthesis. Similarly, in some examples, a prosthesis can take the place of the thumb and resist dorsal movement so as to enable grasping pressure between one or more of digits 120, 130, 140, 150 and the prosthesis.

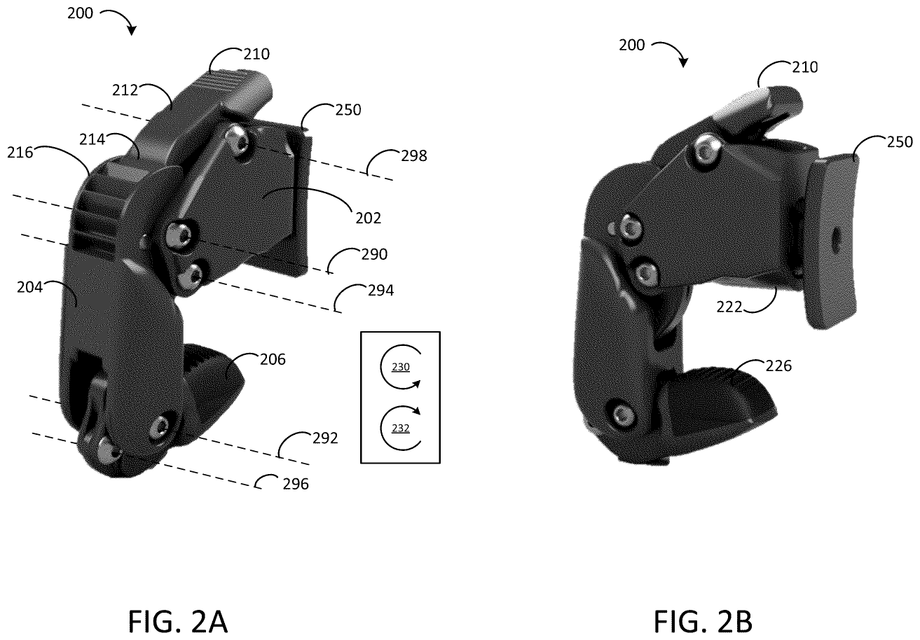

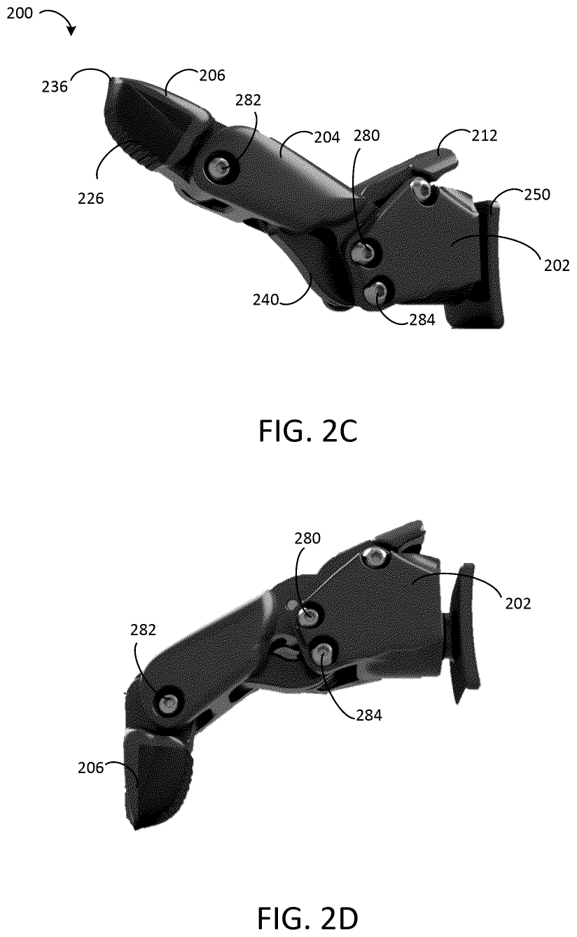

FIG. 2A shows an example prosthetic digit 200. Prosthetic digit 200 includes a proximal segment 202 connected to an intermediate segment 204 about a first axis of rotation 290. In some examples, the proximal segment 202 and intermediate segment 204 are coupled via a pin extending through each component along the first axis of rotation 290 to permit a rotational relationship between the components. The prosthetic digit 200 also includes a distal segment 206 connected to the intermediate segment 204 about a second axis of rotation 292. In some examples, the intermediate segment 204 and distal segment 206 are coupled via a pin extending through each component along the second axis of rotation 292 to permit a rotational relationship between the components.

In some examples, proximal segment 202, intermediate segment 204, and distal segment 206 comprise a hard, lightweight material, such as a plastic or carbon fiber material. In some embodiments, each such segment is made from the same material. In some examples, each of the proximal segment 202, intermediate segment 204, and distal segment 206 include a carbon fiber material. In some embodiments, one or more of the proximal segment 202, intermediate segment 204, and the distal segment 206 can be manufactured by molding, 3D printing, and/or other manufacturing techniques.

In the illustrated example, intermediate segment 204 is shown as rotated downward relative to proximal segment 202. Analogizing the direction of rotation to the hand and digit anatomy discussed with respect to FIG. 1, the intermediate segment 204 can be said to be rotated about the first axis of rotation 290 in a palmar direction (shown by arrow 230). Similarly, the distal segment 206 is shown as being rotated about the second axis of rotation 292 in a palmar direction. Extending the prosthetic digit 200 from the curled configuration shown in FIG. 2A can include rotating the intermediate segment 204 in a dorsal direction (shown by arrow 232) about the first axis of rotation 290 and/or rotating the distal segment 206 in a dorsal direction about the second axis of rotation 292 as discussed elsewhere herein.

In some embodiments, prosthetic digit 200 can include a locking mechanism configured to limit rotation of one segment about an axis of rotation. In some examples, the locking mechanism comprises a unidirectional locking mechanism in which a segment is free to rotate in a first direction about an axis of rotation but rotation in the opposite direction about the same axis of rotation is inhibited by the locking mechanism. In some embodiments, the locking mechanism comprises a unidirectional locking mechanism freely permitting rotation of the intermediate segment 204 in a palmar direction about the first axis of rotation 290 while resisting rotation of the intermediate segment 204 in a dorsal direction about the first axis of rotation 290.

In the example of FIG. 2A, prosthetic digit 200 includes a ratchet surface 216 on the intermediate segment 204 and a lever 212 attached to the proximal segment 202. The lever includes a first end 210 and a second end 214, wherein the second end is configured to act as a pawl relative to the ratchet surface 216. The lever 212 and ratchet surface 216 can act in concert to form the locking mechanism wherein the pawl function of the second end 214 of the lever 212 engages one or more teeth on the ratchet surface 216 of the intermediate segment 204 and resists rotation of the intermediate segment 204 in the dorsal direction about the first axis of rotation 290. However, the locking mechanism of FIG. 2A freely permits rotation of the intermediate segment 204 in the palmar direction about the first axis of rotation 290.

In some embodiments, lever 212 is configured to rotate about axis of rotation 298 in order to selectively engage or disengage pawl at the second end 214 of the lever 212 from the ratchet surface 216. Lever 212 can be spring biased such that the pawl engages the ratchet surface 216 when no outside force is applied. However, depressing first end 210 of the lever 212 can overcome the spring bias and cause the lever to rotate about axis 298 and cause the pawl at the second end 214 of the lever 212 to disengage from the ratchet surface 216. Thus, in some embodiments, depressing the first end 210 of the lever 212 disengages a unidirectional lock and permits free rotation of the intermediate segment 204 in both the palmar and dorsal directions about the first axis of rotation 290.

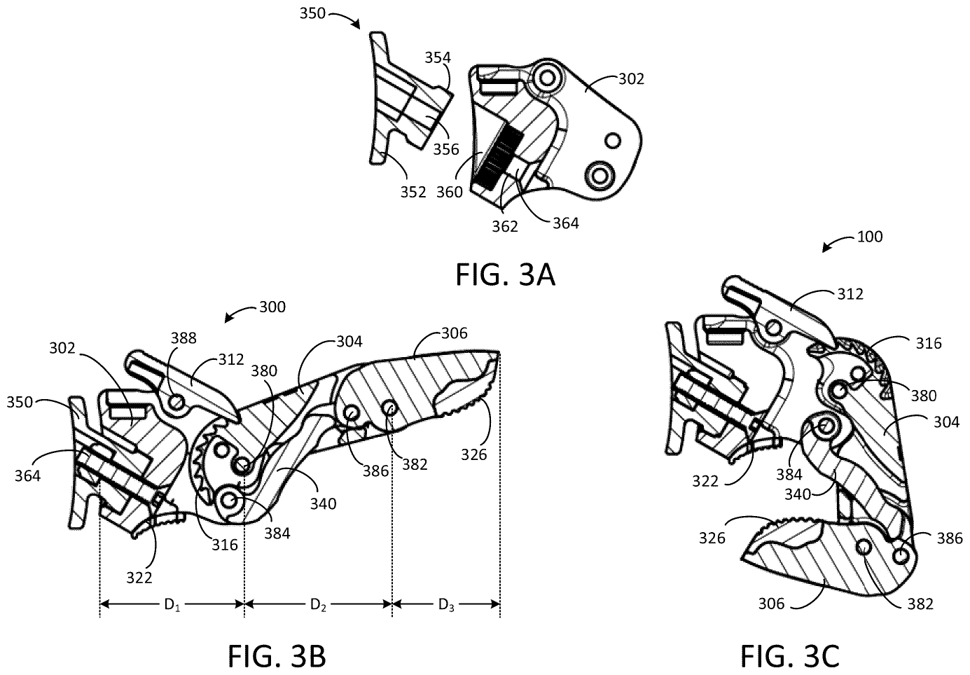

In some examples, a prosthetic digit includes a linkage coupled to the proximal segment 202 and the distal segment 206. An example linkage is discussed in greater detail with respect to FIGS. 2C and 2D as well as FIGS. 3A-3C. The linkage can be configured to cause a distal end of the distal segment to rotate in a palmar direction about the second axis 292 when the intermediate segment 204 is rotated in a palmar direction about the first axis of rotation 290. In some such examples, palmar rotation of the intermediate segment 204 about the first axis of rotation 290 forces the linkage to rotate in the palmar direction about third axis of rotation 294. Combined palmar rotation of the intermediate segment 204 about first axis of rotation 290 and linkage about the third axis of rotation 294 can force relative movement between an attachment point between linkage and the distal segment 206 (along axis 296) and an attachment point between the intermediate segment 204 and the distal segment 206 (along axis 292). Such movement can cause the distal segment 206 to rotate in a palmar direction about the second axis of rotation 292.

In some examples, the pawl and ratchet surface configuration of the lever 212 and intermediate segment 204 creates a plurality of discrete rotational positions for the intermediate segment 204 about the first axis of rotation 290. In some examples, a linkage creates a 1:1 relationship between unique rotational positions of the intermediate segment 204 about the first axis of rotation 290 and corresponding unique rotational positions of the distal segment 206 about the second axis of rotation 292. Thus, in some embodiments, the prosthetic digit 200 can be locked into a plurality of discrete configurations wherein each configuration corresponds to a unique rotational position of the intermediate segment 204 about the first axis of rotation 290 and of the distal segment 206 about the second axis of rotation 292.

The prosthetic digit 200 of FIG. 2A is shown with an anchor 250. In some embodiments, the prosthetic digit 200 is removable from anchor 250. In some examples, anchor 250 can be fixedly attached to a wearer or to a wearable support configured to be worn by the user. In some such embodiments, a prosthetic digit such as prosthetic digit 200 can be removably attached to the anchor 250 in order to attach the prosthetic digit to the wearer.

FIG. 2B shows an alternate view of the prosthetic digit 200 shown in and described with respect to FIG. 2A. In some embodiments, the distal segment 206 includes a fingertip surface 226 on a palmar surface of the distal segment 206. In some examples, the fingertip surface 226 can be made from a different material from the distal segment, 206, such as a more compliant material, for example, a complaint rubber material. A compliant fingertip surface 226 can provide a firm and/or non-slip gripping surface for the prosthetic digit 200 to assist in grasping an object.

In some examples, the proximal segment 202 includes a base pad surface 222 positioned on the palmar side of the proximal segment 202. In some embodiments, the base pad surface 222 comprises a more compliant material than the proximal segment. For example, in some embodiments, base pad surface 222 comprises a compliant rubber. A compliant base pad surface 222 can provide a firm and/or non-slip gripping surface for the prosthetic digit 200 to assist in grasping an object.

In some embodiments, prosthetic digit 200 is configured such that, as the prosthetic digit articulates in a palmar direction (e.g., intermediate segment 204 rotates in a palmar direction about the first axis of rotation 290 and the distal segment 206 rotates in a palmar direction about the second axis of rotation 292) fingertip surface 226 generally moves toward base pad surface 222, as shown in FIG. 2B. In some examples, such movement creates a clamping action between the fingertip surface 226 and the base pad surface 222. A locking mechanism resisting dorsal movement of the prosthetic digit can operate in conjunction with the fingertip surface 226 and the base pad surface 222 to create a clamp-like mechanism whereby the fingertip surface 226 and base pad surface 222 can clamp to and hold an object in the space therebetween. In some examples, one or both of the fingertip surface 226 and base pad surface 222 includes a curved surface. In other examples, one or both of the fingertip surface 226 and the base pad surface 222 are flat surfaces.

FIGS. 2C and 2D show side views of the prosthetic digit 200 wherein the intermediate segment 204 and the distal segment 206 are in different rotational positions. In the example of FIG. 2C, prosthetic digit is in a fully-extended state. Compared to the curled state shown in FIGS. 2A and 2B, the intermediate segment 204 has rotated in a dorsal direction about axis 290 and the distal segment 206 has rotated in a dorsal direction about axis 292. In some embodiments, the fully-extended configuration of the prosthetic digit 200 shown in FIG. 2C is a default position assumed by the prosthetic digit 200 when a releasable unidirectional locking mechanism (e.g., lever 212 and ratchet surface 216) is released. Such a default position may be achieved, for example, by spring biasing the intermediate segment 204 to rotate in a dorsal direction about axis 290. In some examples, the spring bias is created by an extension spring internal to the prosthetic digit and providing a downward pull on a proximal portion of the intermediate segment 204 toward a portion of the proximal segment 202.

As shown in FIG. 2C, proximal segment 202 can be attached to an anchor 250 to facilitate attaching prosthetic digit 200 to a wearer. The proximal segment 202 can be attached to an intermediate segment 204 via a pin 280 defining an axis of rotation (e.g., first axis of rotation 290 in FIG. 2A) such that the intermediate segment 204 can rotate about the pin 280. Intermediate segment 204 can be attached to distal segment 206 via a pin 282 defining an axis of rotation (e.g., second axis of rotation 292 in FIG. 2A) such that the distal segment 206 can rotate about the pin 282.

As discussed above, in some examples, distal segment 206 includes a fingertip surface 226 positioned on a palmar surface of the distal segment 206. Additionally or alternatively, in some examples, the distal segment 206 includes a fingernail portion 236 protruding from a dorsal surface of the distal segment 206. The fingernail portion 236 can facilitate using the prosthetic digit 200 to pick up or grasp small or thin objects, for example, picking up coins or other relatively flat objects.

In some embodiments, prosthetic digit 200 includes a linkage 240 coupled to the proximal segment 202 and the distal segment 206. In the illustrated example, linkage 240 is coupled to proximal segment 202 via pin 284, which can define an axis of rotation (e.g., axis of rotation 294 shown in FIG. 2A). Linkage 240 can be coupled to the distal segment 206 at a position not shown in FIG. 2C such that, as the intermediate segment 204 is rotated in a palmar direction about a rotational axis (e.g., defined at pin 280), linkage 240 rotates in a palmar direction about a different rotational axis (e.g., defined at pin 284) and pushes on an attachment point of the distal segment 206 so as to cause the distal segment to rotate in a palmar direction about yet another rotation axis (e.g., defined at pin 282).

As shown in the example of FIG. 2C, each of the proximal segment 202, the intermediate segment 204, and the distal segment 206 add a distal projection length away from the anchor 250. In some embodiments, projecting an axis of rotation 290 away from the anchor 250 by proximal segment 202 offsets the axis of rotation of the intermediate segment 204 from the attachment point of the prosthetic digit 200 to the anchor. The prosthetic digit can be configured such that, as the prosthetic digit 200 rotates in a palmar direction, the intermediate segment 204 shifts forward relative to the proximal segment 202 while the proximal segment 202 remains attached to the anchor. In some examples, rotation of the intermediate segment 204 about the axis of rotation 290 causes the intermediate segment 204 to shift forward relative to the proximal segment 202 without the proximal segment 202 shifting.

FIG. 2D shows a prosthetic digit 200 in an intermediate state. In the example of FIG. 2D, the intermediate segment 204 of the prosthetic digit 200 is rotated about an axis defined by pin 280 in a palmar direction relative to the configuration shown in FIG. 2C. Similarly, the distal segment 206 of the prosthetic digit 200 is rotated about an axis defined by pin 282 in a palmar direction relative to the configuration shown in FIG. 2C.

In the illustration of FIG. 2D, linkage 240 has rotated in a palmar direction about an axis defined by pin 284. In some examples, linkage 240 is coupled to the proximal segment 202 at pin 284 and is coupled to the distal segment 206. The prosthetic digit 200 of FIG. 2D can be configured such that rotation of the linkage 240 about axis defined by pin 284 and rotation of the intermediate segment 204 about an axis defined by pin 280 can cause the distal segment 206 to rotate about an axis defined by pin 282. This can be caused by the relationship between attachment and rotation points between (i) the linkage 240 and the proximal segment 202 (e.g., at pin 284), (ii) the intermediate segment 204 and the proximal segment 202 (e.g., at pin 280), (iii) the intermediate segment 204 and the distal segment 206 (e.g., at pin 282), and the linkage 240 and the distal segment 206 (e.g., at a point intersecting axis 296 shown in FIG. 2A and not shown in FIG. 2D).

In some examples, a prosthetic digit is positionable to a plurality of configurations between a fully-extended configuration (e.g., the configuration shown in FIG. 2C) and a fully-closed configuration. In some cases, a fully-closed configuration corresponds to a configuration in which the intermediate segment 204 is maximally rotated in the palmar direction about rotational axis 290. In some embodiments, the fully-closed configuration corresponds to a configuration in which the fingertip surface 226 engages the base pad surface 222. In other embodiments, the fully-closed configuration does not include the fingertip surface 226 engaging the base pad surface 222, but instead includes a gap between the fingertip surface 226 and the base pad surface 222. For example, in some embodiments, the configuration shown in FIG. 2A or 2B is a fully-closed configuration.

FIGS. 3A-3C show cross-sectional views of the example prosthetic digit and anchor of FIG. 2. FIG. 3A shows an example attachment of a proximal segment 302 of a prosthetic digit to an anchor 350. In the illustrated example, anchor 350 includes a plate 352 and a flange 354 protruding therefrom. Flange 354 includes a receptacle 356.

In the example of FIG. 3A, the proximal segment of a proximal segment 302 of the prosthetic digit includes a receptacle 360 in proximal side of the proximal segment 302 sized to receive the flange 354 of the anchor 350. The proximal segment 302 further includes a channel 362 extending through from a distal side of the proximal segment 302 to the receptacle 360. As shown, a removable fastener 364 is positioned within the channel 362. In the illustrated example, removable fastener 364 comprises a screw or bolt, however, in various embodiments other fasteners could be used, such as a bayonet mount fastener.

In some examples, fastener 364 comprises a threaded post configured to extend through channel 362 in the proximal segment 302. In some such embodiments, receptacle 356 in the flange 354 of the anchor 350 comprises complementary threads such that the threaded post of fastener 364 can be securely fastened to receptacle 356. In some embodiments, receptacle 356 is itself threaded. In other examples, receptacle 356 can house a nut or other threaded components therein that is configured to threadably engage a threaded post of the fastener. Thus, in some embodiments, a prosthetic digit can be attached to the anchor 350 by inserting the flange 354 of the anchor 350 into the receptacle 360 of the proximal segment 302 and securing the proximal segment 302 to the anchor 350 by threading a fastener 364 into receptacle 356 of the flange.

Additionally, as noted above, in some embodiments, the fastener 364 is removable from the receptacle 356 of the anchor 350. In such cases, once a prosthetic digit is mounted to the anchor 350, the prosthetic digit can be removed, for example, for repair or replacement or any other reason. Additionally or alternatively, if the prosthetic digit is misaligned, the prosthetic digit can be adjusted relative to the anchor 350.

In some examples, the flange 354 of the anchor 350 and the receptacle 360 of the proximal segment 302 are complementary in shape. In some cases, each comprises a circular shape. In some embodiments, the receptacle 360 can receive the flange 354 in a plurality of rotational orientations. In some cases, the complementary shapes are non-circular, but rather have complementary edges, points, grooves, or some other shape to permit a discrete number of rotational orientations (e.g., complementary square flange 354 and receptacle 360 could allow for up to four discrete rotational orientations). In some embodiments, an inner surface of the receptacle 360 and an outer surface of flange 354 are serrated in order to permit a finite number of orientations. In other examples, a continuum of rotational orientations is possible (e.g., if the complementary shapes are circular and do not require one of a plurality of discrete orientations). In either case (discrete or continuous rotational positions), in some embodiments, rotational orientation may be limited by other design factors, for example, restricting rotation to a finite amount of rotation from a base orientation (e.g., ±5°, ±10°, ±15°, ±30°, ±45°).

In some examples, such attachment and detachment ability can be used to fit a prosthetic digit to a wearer. In an example process, a prosthetic digit can be attached to an anchor affixed to a wearer (e.g., via a wearable support to which the anchor is affixed). Attaching the prosthetic digit can include engaging a threaded fastener 364 to receptacle 356 of the flange 354 of the anchor 350, but not securing the fastener 364 fully so as to permit movement of the prosthetic digit relative to the anchor 350. The process can include rotating the prosthetic digit about an axis relative to the anchor, for example, until the prosthetic digit is arranged in a target rotational position. In some examples, a target rotational position includes an orientation permitting the prosthetic digit to properly engage with an existing digit, such as a thumb, or to cooperate with another prosthetic digit. In some examples, the target orientation is determined empirically by rotating the prosthetic digit until a suitable orientation is found. The process can further include, once the prosthetic digit is rotated to the target rotational position, securing the prosthetic digit to the anchor in the target rotational position. Securing the prosthetic digit to the anchor can include tightening the threaded fastener 364 to prevent movement of the prosthetic digit relative to the anchor.

FIG. 3B shows a cross section of a prosthetic digit attached to an anchor. In the example, prosthetic digit 300 comprises a proximal segment 302 removably attached to anchor 350 via fastener 364. The proximal segment 302 is attached to intermediate segment 304 at a pin 380 defining an axis of rotation about which intermediate segment 304 can rotate relative to proximal segment 302 such as described elsewhere herein. Intermediate segment 304 is attached to distal segment 306 at a pin 382 defining an axis of rotation about which distal segment 306 can rotate relative to intermediate segment 304 such as described elsewhere herein.

The prosthetic digit 300 includes a unidirectional locking mechanism comprising a lever 312 acting as a pawl engaging a ratchet surface 316 of the intermediate segment 304. The intermediate segment 304 is permitted to rotate freely in a palmar direction about an axis defined at pin 380, but is prevented from rotating in the dorsal direction, opposite the palmar direction, when pawl engages ratchet surface 316. In some embodiments, lever 312 is spring biased so that pawl engages the ratchet surface 316 unless the spring bias is overcome. In some embodiments, lever is manipulable to overcome the spring bias to disengage pawl from the rachet surface 316. In some examples, lever can rotate about an axis defined at pin 388 such that if a side of the lever opposite the pawl is depressed, the pawl disengages from the ratchet surface 316 and the intermediate segment 304 can rotate freely in both the dorsal and palmar directions.

In the example of FIG. 3, a linkage 340 is coupled to the proximal segment 302 at pin 384 and to the distal segment 306 at pin 386 and is configured to rotate relative to such components about axes defined by such pins. The linkage 340 can be configured such that rotation of the intermediate segment 304 in the palmar direction about an axis defined by pin 380 causes rotation of the linkage 340 about an axis defined by pin 384. Such rotation and the relative position of pins can cause the distal segment 306 to rotate about an axis defined by pin 382 in response to the rotation of intermediate segment 304 about the axis defined by pin 380.

The example of FIG. 3B shows a fingertip surface 326 positioned on a palmar surface of the distal segment 306 and a base pad surface 322 positioned on a palmar surface of proximal segment 302. As described elsewhere herein, in some embodiments, fingertip surface 326 and base pad surface 322 can be made from a more compliant material compared to other portions of the proximal segment 302 and the distal segment 306.

As described above, in some examples, each of proximal segment 302, intermediate segment 304, and distal segment 306 extend away from the anchor 350 when the prosthetic digit is in an extended configuration, such as shown in FIG. 3B. In some embodiments, when the prosthetic digit is in an extended configuration, the proximal segment 302 extends an axis of rotation about pin 380 a first distance D₁ from the anchor 350, and intermediate segment 304 extends an axis of rotation about pin 382 a distance D₂ from the axis about pin 380. In the illustrated example, a distal end of the distal segment 306 is a distance D₃ from the axis of rotation about pin 382. In some examples, D₁, D₂, and D₃ are approximately equal in length. In other examples, D₂>D₁>D₃. In some embodiments, Each of D₁, D₂, and D₃ are between 0.25 inches and 1.5 inches.

FIG. 3C shows a cross-section of a prosthetic digit rotated in a palmar direction compared to the configuration in FIG. 3B. In the illustrated examples, intermediate segment 304 is rotated in a palmar direction about an axis defined by pin 380 compared to the configuration in FIG. 3B. Such rotation, along with rotation of linkage 340, coupled to proximal segment 302 via pin 384 and to distal segment 306 via pin, about an axis defined by pin 384, causes the distal segment 306 to rotate in a palmar direction about axis defined by pin 382. In some examples, the axis of rotation defined by pin 380 is offset from a central longitudinal axis of the intermediate segment 304. In some cases, rotation of the intermediate segment 304 about the axis defined by pin 380 causes a portion of the intermediate segment 304 to move distally. In some embodiments, such distal motion contributes to the causal relationship between rotation of the intermediate segment 304 about the axis defined by pin 380 and rotation of the distal segment 306 about the axis defined by pin 382.

As described elsewhere herein, in some embodiments, the prosthetic digit is configured such that, as the prosthetic digit 300 rotates in a palmar direction, the intermediate segment 304 shifts forward relative to the proximal segment 302 while the proximal segment 302 remains attached to the anchor. In some examples, rotation of the intermediate segment 304 about the axis defined by pin 380 causes the intermediate segment 304 to shift forward relative to the proximal segment 302 without the proximal segment 302 shifting. In some embodiments, the offset of the axis of rotation defined by pin 380 relative to a central longitudinal axis of the intermediate segment 304 contributes to such a distal shift of the intermediate segment 304 without shifting the proximal segment 302.

As shown, in the configuration of FIG. 3C, fingertip surface 326 is moved toward base pad surface 322 as the prosthetic digit articulates in a palmar direction. In some embodiments, fingertip surface 326 and base pad surface 322 form a clamping mechanism to assist in grasping an object with the prosthetic digit. As shown in the cross-sectional view of FIG. 3C, pawl of lever 312 engages ratchet surface 316 of intermediate segment 304, preventing dorsal rotation of the intermediate segment 304 about the axis defined by pin 380. In some embodiments, the linked relationship between intermediate segment 304 and distal segment 306 prevents dorsal rotation of the distal segment 306 about the axis defined by pin 386 if the intermediate segment 304 is prevented from dorsal rotation about the axis defined by pin 380. Thus, in some embodiments, the locking mechanism, when engaged, prevents motion of the fingertip surface 326 away from the base pad surface 322.

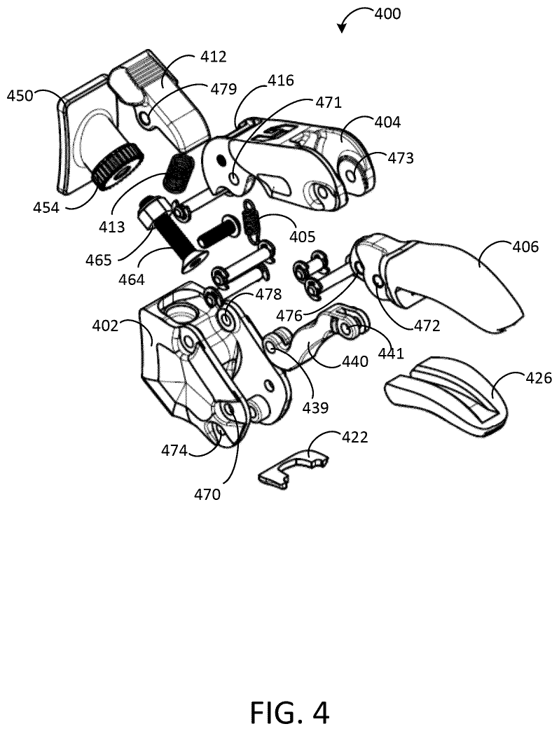

FIG. 4 shows an exploded view of a prosthetic digit according to some embodiments. The example of FIG. 4 includes a prosthetic digit 400 having a proximal segment 402 couplable to anchor 450. A threaded fastener 464 extends through a section of the proximal segment 402 and engages a threaded nut 465, which is positioned in flange 454 of the anchor 450. In some embodiments, securing the threaded fastener 464 to threaded nut 465 while flange 454 of the anchor engages a receptacle of the proximal segment 402 secures the proximal segment 402 to the anchor 450.

The proximal segment 402 is configured to connect to intermediate segment 404 at a first axis of rotation, which can be located where hole 470 of the proximal segment 402 aligns with hole 471 of the intermediate segment 404. As shown, hole 471 of the intermediate segment 404 is offset from a central longitudinal axis of the intermediate segment 404, which can cause one or more components of the prosthetic digit 400 to move distally as the intermediate segment 404 rotates about the first axis of rotation.

The prosthetic digit 400 includes a distal segment 406 configured to connect to the intermediate segment 404 about a second axis of rotation, which can be located where hole 472 of distal segment 406 aligns with hole 473 of the intermediate segment 404. A linkage 440 is configured to couple the proximal segment 402 to the distal segment 406, and can be connected to the proximal segment at an axis of rotation, which can be located where hole 474 of the proximal segment 402 and hole 439 of linkage 440 align. Linkage can connect to distal segment 406 at a location where hole 441 of linkage and hole 476 of distal segment 406 align. In some embodiments, the linkage is configured to cause a distal end of the distal segment to rotate toward the proximal segment about the second axis of rotation when the intermediate segment is rotated in a palmar direction about the first axis of rotation.

The prosthetic digit 400 includes a lever 412 configured to act as a pawl and engage ratchet surface 416 of the intermediate segment 404 as described elsewhere herein to form a unidirectional lock. Lever can be connected to intermediate segment 404 at a location where hole 479 of the lever and hole 478 of the intermediate segment 404 align. Lever 412 can be spring biased into a locking position, in which pawl engages ratchet surface 416, by a spring 413, which can bias a proximal end of the lever 412 upward to hold the pawl against the ratchet surface 416. The proximal end of the lever 412 can be depressed to rotate the lever 412 about the aligned holes 478, 479 to disengage pawl from ratchet surface 416 and release the unidirectional lock.

As described herein, in some examples, the unidirectional lock formed by pawl and ratchet surface 416 resist dorsal rotation of intermediate segment 404. In some embodiments, intermediate segment 404 is spring biased to rotate in the dorsal direction about an axis where holes 470 and 471 meet such that action of the unidirectional lock resists against the spring bias. Such spring bias can be created by spring 405, which can include an extension spring that pulls a proximal end of the intermediate segment 404 toward a portion of the proximal segment 402 to cause dorsal rotation of the intermediate segment 404.

The exploded view of FIG. 4 further shows a fingertip surface 426 configured to attach to a palmar side of distal segment 406 and a base pad surface 422 configured to attach to a palmar side of the proximal segment 402.

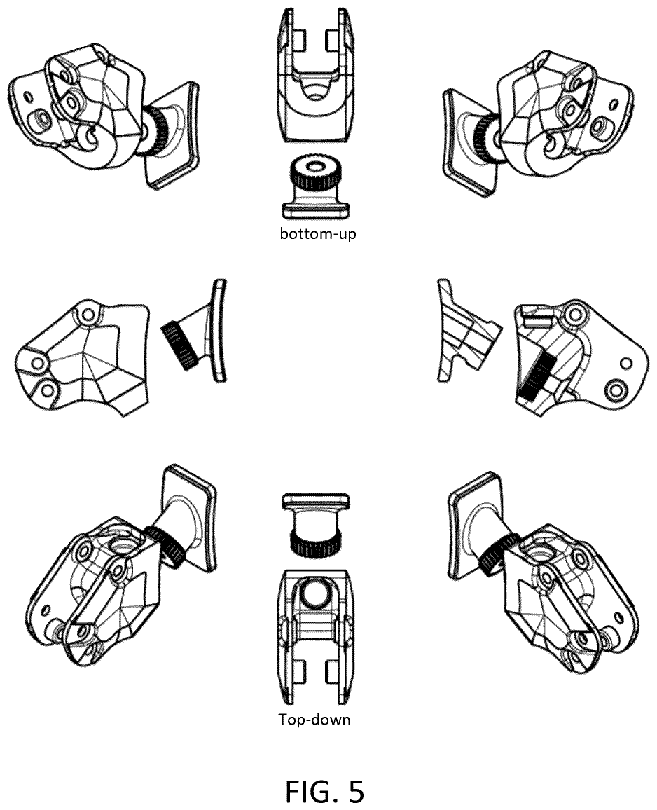

FIG. 5 shows a plurality of views of an anchor and a proximal segment of a prosthetic digit configured to attach to the anchor. FIG. 5 includes a top-down view, bottom-up view, a side view, a plurality of perspective views, and a cross-sectional view of an example embodiment.

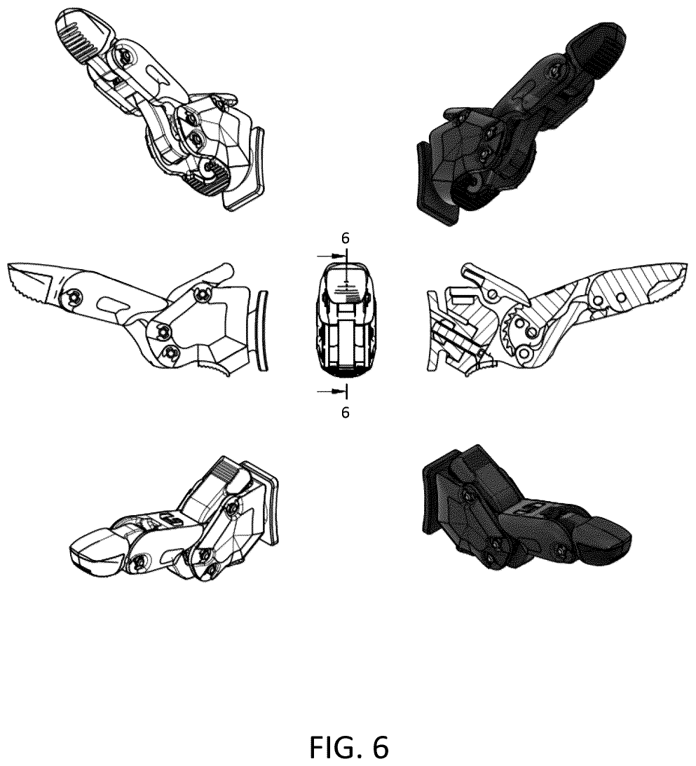

FIG. 6 shows a plurality of views of an example prosthetic digit in an extended configuration and attached to an anchor. FIG. 6 shows a plurality of perspective views, a side view, a front-on view, and a cross-sectional view taken along line 6-6 of the front-on view.

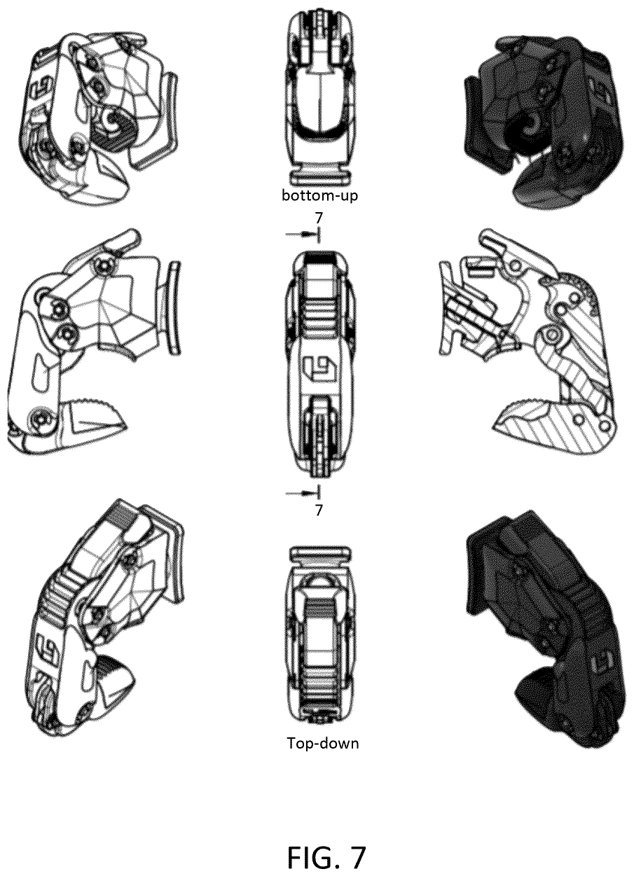

FIG. 7 shows a plurality of views of an example prosthetic digit in a curled configuration and attached to an anchor. FIG. 7 shows a plurality of perspective views, a top-down view, a bottom-up view, a side view, a front-on view, and a cross-sectional view taken along line 7-7 of the front-on view.

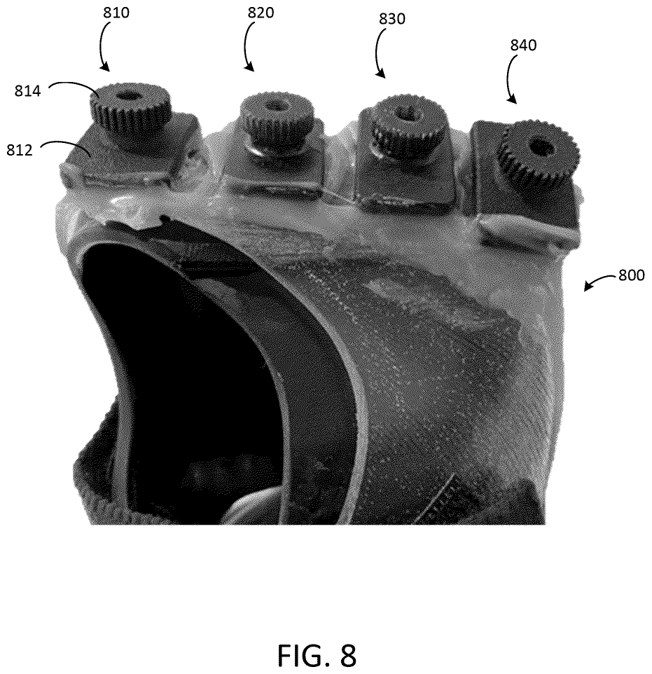

FIG. 8 shows a wearable support configured to be attached to a wearer. In various examples, wearable support is configured to be securely fastened to one or both of a wearer's hand or wrist, such as via one or more straps or the like. In the illustrated example, wearable support 800 holds a plurality of anchors 810, 820, 830, 840, each configured to receive a prosthetic digit to affix to the wearable support via the anchor. In various embodiments, the wearable support can be sized to support any of one, two, three, four, or more anchors. For example, a custom wearable support can be created to provide an anchor at a location of a digit that has been amputated at or proximal to an MCP joint. In some examples, one or more such anchors are permanently affixed to the wearable support 800, such as via lamination, an adhesive attachment, or other attachment processes. In some examples, an anchor is affixed to the wearable support via an anchor plate of the anchor, such as via anchor plate 812 of anchor 810 in FIG. 8. In some embodiments, the anchor plate 812 can secure the anchor 810 to the wearable support 800 such that a prosthetic digit can be affixed to the anchor 810 via a flange 814 attached to the anchor plate 812, such as described elsewhere herein.

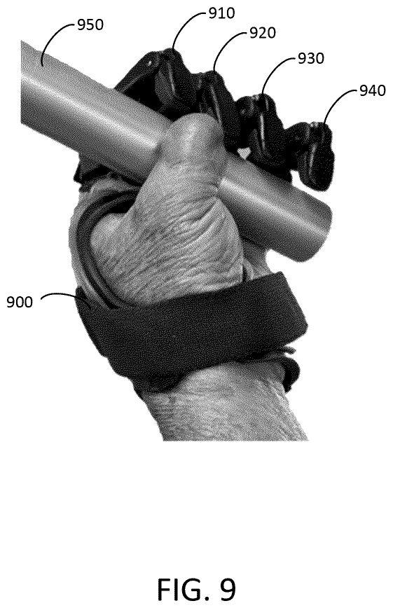

FIG. 9 shows a wearable support attached to a wearer's anatomy and supporting a plurality of prosthetic digits. As shown, wearable support 900 is attached to a wearer's hand and wrist region. As shown, wearable support 900 supports prosthetic digits 910, 920, 930, and 940. As discussed elsewhere herein, in some examples, the wearable support 900 includes a plurality of anchors configured to support a corresponding plurality of prosthetic digits 910, 920, 930, and 940. In some embodiments, the rotational position of one or more prosthetic digits 910, 920, 930, and 940 can be adjusted relative to the anchor to which it is mounted in order to adjust the alignment of the prosthetic digit to another object, such as a wearer's anatomy.

In the example of FIG. 9, prosthetic digits 910, 920, 930, and 940 are supported by a wearable support 900 secured to a wearer's anatomy. As shown, prosthetic digits 910, 920, 930, and 940 are in a curled position and assist the wearer in grasping object 950. As described herein, in some embodiments, a prosthetic digit includes a unidirectional locking mechanism permitting rotation of one or more components in a palmar direction (in the illustrated example, to close/curl the prosthetic digit), and preventing rotation in the opposite, dorsal direction. In such case, a prosthetic digit such as shown in FIG. 9 can be used to securely grasp object 950 without opening due to the unidirectional lock resisting dorsal rotation of the prosthetic digit.

In some embodiments, to engage a prosthetic digit in the closed configuration, the prosthetic digit can be manually rotated in the palmar direction without obstruction by the unidirectional lock until the prosthetic digit reaches a desired position. Once at the desired position, the unidirectional lock prevents undesired dorsal rotation, permitting the prosthetic digit to assist in grasping and/or functions.

In some embodiments, prosthetic digits described herein can utilize one or more features described in U.S. Pat. No. 11,311,393 (“the '393 patent”), granted Apr. 26, 2022, and entitled UNIVERSAL DIGIT, the entire contents of which is incorporated herein by reference. For example, one or more segments can longitudinally rotatable relative to a segment attached thereto. In some examples, an intermediate segment can be attached to a proximal segment via configurations described in the '393 patent. Additionally or alternatively, a proximal segment can be attached to an anchor via configurations described in the '393 patent. Additionally or alternatively, a distal segment can be attached to an intermediate segment via configurations described in the '393 patent. For example, in some embodiments, one segment may be rotatably coupled to an adjacent segment wherein a button mechanism can enable repositioning of one segment relative to the adjacent segment when the button mechanism is depressed, and repositioning in such a manner is prevented after the button is released.

Various examples have been described. These and others are within the scope of the following claims.

1. A prosthetic digit comprising:
a proximal segment connected to an intermediate segment about a first axis of rotation;
a distal segment connected to the intermediate segment about a second axis of rotation;
a linkage coupling the proximal segment to the distal segment and being connected to the proximal segment about a third axis of rotation, the linkage being configured to cause a distal end of the distal segment to rotate toward the proximal segment about the second axis of rotation when the intermediate segment is rotated in a palmar direction about the first axis of rotation; and
a locking system comprising a unidirectional lock and a release mechanism, the unidirectional lock being configured to allow the intermediate segment to rotate freely in the palmar direction about the first axis of rotation but locking the intermediate segment from rotating in a dorsal direction, the release mechanism being configured to release the unidirectional lock to enable rotation of the intermediate segment in the dorsal direction.

2. The prosthetic digit of claim 1, wherein the intermediate segment is spring biased in the dorsal direction about the first axis of rotation such that rotating the intermediate segment in the palmar direction overcomes the spring bias.

3. The prosthetic digit of claim 2, wherein, when the release mechanism releases the unidirectional lock, the spring bias of the intermediate segment causes the intermediate segment to rotate in the dorsal direction.

4. The prosthetic digit of claim 1, wherein the unidirectional lock comprises a ratchet surface positioned on the intermediate segment and a pawl configured to engage the ratchet surface and prevent rotation of the intermediate segment in the dorsal direction.

5. The prosthetic digit of claim 4, wherein the release mechanism comprises a lever coupled to the proximal segment, the lever having a first end and a second end, and wherein the pawl of the unidirectional lock is formed by the second end of the lever.

6. The prosthetic digit of claim 5, wherein:
the lever is spring biased in favor of the pawl engaging the ratchet surface of the intermediate segment; and
the lever is manipulable to overcome the spring bias and disengage the pawl from the ratchet surface to release the unidirectional lock to enable rotation of the intermediate segment in the dorsal direction.

7. The prosthetic digit of claim 6, wherein the first end of the lever is positioned to be depressed to overcome the spring bias of the lever and disengage the pawl from the ratchet surface.

8. The prosthetic digit of claim 7, wherein the first end of the lever is configured to be depressed in the palmar direction thereby causing the pawl to move in the dorsal direction.

9. The prosthetic digit of claim 7, wherein the intermediate segment is spring biased in the dorsal direction about the first axis of rotation.

10. The prosthetic digit of claim 4, wherein the intermediate segment comprises a curved surface on a dorsal side of the intermediate segment, and the rachet surface comprises a plurality of teeth at spaced locations along the curved surface.

11. The prosthetic digit of claim 10, wherein the plurality of teeth define a plurality of discrete locking positions of the intermediate segment.

12. The prosthetic digit of claim 1, further comprising an anchor, wherein the proximal segment is configured to removably connect to the anchor.

13. The prosthetic digit of claim 12, wherein the proximal segment is configured to be removably coupled to the anchor in a plurality of rotational positions.

14. The prosthetic digit of claim 1, wherein the distal segment includes a fingertip surface on a palmar side of the distal segment.

15. The prosthetic digit of claim 14, wherein the proximal segment comprises a base pad surface positioned on a palmar surface of the proximal segment.

16. The prosthetic digit of claim 15, wherein the intermediate segment and the distal segment are configured to rotate in the palmar direction to close a separation distance between the fingertip surface and the base pad surface.

17. The prosthetic digit of claim 15, wherein the proximal segment, the intermediate segment, and the distal segment are made from a first material and wherein the fingertip surface and the base pad surface are each made from a material more compliant than the first material.

18. The prosthetic digit of claim 1, wherein the distal segment includes a fingernail segment protruding from an end of the distal segment.

19. A prosthetic digit comprising:
a proximal segment having a lever, the lever having a first end and a second end and being pivotable about a first axis of rotation between a first position and a second position and being spring biased toward the first position such that the spring bias opposes pivoting the lever about the first axis of rotation in the direction from the first position toward the second position and depressing the first end of the lever overcomes the spring bias to pivot the lever toward the second position;
an intermediate segment having a proximal end and a distal end, the proximal end of the intermediate segment being pivotably attached to the proximal segment and having a plurality of teeth arranged about an outer edge of the proximal end forming a ratchet surface, the intermediate segment being coupled to the proximal segment such that the intermediate segment is pivotable relative to the proximal segment about a second axis of rotation;
wherein the intermediate segment is pivotable between a fully-extended position and a fully-closed position;
the intermediate segment is spring-biased toward the fully-extended position;
the lever of the proximal segment engages one or more of the teeth arranged about the outer edge of the proximal end of the intermediate segment when the lever is in the first position and does not engage any of the teeth when the lever is in the second position such that:
the lever of the proximal segment resists the intermediate segment pivoting toward the fully-extended position when engaging one or more teeth about the outer edge of the proximal end of the intermediate segment; and
when the first end of the lever is depressed, the lever pivots to the second position and the intermediate segment pivots toward the fully-extended position.

20. The prosthetic digit of claim 19, wherein the intermediate segment is configured to rotate in a palmar direction from the fully-extended position toward the fully-closed position.

21. The prosthetic digit of claim 19, wherein the intermediate segment includes a central axis extending longitudinally from the proximal end to the distal end, and wherein the second axis of rotation is offset from the central axis of the intermediate segment.

22. The prosthetic digit of claim 19, further comprising a distal segment pivotably attached to the distal end of the intermediate segment such that the distal segment is capable of pivoting relative to the intermediate segment about a third axis of rotation.

23. The prosthetic digit of claim 22, further comprising a linkage coupled to the proximal segment and the distal segment such that pivoting the intermediate segment about the second axis of rotation causes the distal segment to pivot about the third axis of rotation.

24. The prosthetic digit of claim 23, wherein the distal segment includes fingertip surface.

25. The prosthetic digit of claim 24, wherein the proximal segment comprises a base pad surface positioned on a surface of the proximal segment.

26. The prosthetic digit of claim 25, wherein the intermediate segment is configured to pivot from the fully-extended position toward the fully-closed position to close a separation distance between the fingertip surface and the base pad surface.

27. The prosthetic digit of claim 26, wherein, when the intermediate segment is in the fully-closed position, a gap exists between the fingertip surface and the base pad surface.

28. The prosthetic digit of claim 25, wherein the proximal segment, the intermediate segment, and the distal segment are made from a first material and wherein the fingertip surface and the base pad surface are each made from a material more compliant than the first material.

29. The prosthetic digit of claim 22, wherein the distal segment includes a fingernail portion protruding from an end of the distal segment.
